(19) United States
(12) Patent Application Publication
(10) Pub. No.: US 2026/0000457 A1
(43) Pub. Date: Jan. 1, 2026

(54) DEVICE OF INTEGRATING EXTERNAL COORDINATE SYSTEM AND BRAIN MODEL TO GENERATE BIOPSY PATH AND METHOD THEREOF

(71) Applicant: NATIONAL YANG MING CHIAO TUNG UNIVERSITY, Hsinchu City (TW)

(72) Inventors: Shin Tai CHONG, Hsinchu City (TW); Kuan-Tsen KUO, Hsiuchu City (TW); Yu-Ting KO, Hsinchu City (TW); Chun-Yi LO, Hsinchu City (TW); Ching-Po LIN, Hsinchu City (TW)

(21) Appl. No.: 19/234,081

(22) Filed: Jun. 10, 2025

(30) Foreign Application Priority Data
Jun. 27, 2024 (TW) 113123941

(51) Int. Cl. A61B 34/10; G06T 7/00; G06T 7/11; G06T 7/30; G06T 17/00

(52) U.S. Cl. CPC A61B 34/10; G06T 7/0012; G06T 7/11; G06T 7/30; A61B 2034/105; A61B 2034/107; G06T 17/00; G06T 2207/30016; G06T 2207/30101; G06T 2210/41

(57) ABSTRACT

A device of integrating an external coordinate system and a brain model to generate a biopsy path and a method thereof are disclosed. In the device, through registration of brain images of a target person, a registration integration image is generated, binary mask images of different cerebral tissues are extracted from the registration integration image, 3D models are reconstructed based on the binary mask images of the cerebral tissues, an external coordinate system on a skin surface model in the 3D models is established; after a target area in the 3D models is selected and a biopsy point is set, a biopsy path in the 3D models is calculated based on position information of the target area and a coordinate of the biopsy point in the external coordinate system, and the biopsy path in the 3D models is marked.

### CROSS-REFERRENCE STATEMENT

The present application is based on, and claims priority from, TAIWAN Patent Application Serial Number 113123941, filed Jun. 27, 2024, the disclosure of which is hereby incorporated by reference herein in its entirety.

### BACKGROUND

### 1. Technical Field

The present invention is related to a device for generating a biopsy path for a brain and a method thereof, and particularly to a device of integrating an external coordinate system and a brain model to generate a biopsy path and a method thereof.

### 2. Description of Related Arts

In recent years, medical images can provide structural information of a brain, such as white matter, gray matter, blood vessels, cerebrospinal fluid and are widely used in disease diagnosis, intraoperative navigation, and postoperative evaluation of patients. The medical images can be 3D images obtained by a non-invasive manner (such as magnetic resonance imaging MRI) or computed tomography (CT)) through adjustments of different parameters and sequences.

With the rapid advancement of technology, the breakthrough in computing power, and the increasing popularity of precision medicine and personalized minimally invasive surgery, surgical planning has become a crucial aspect of successful surgery. Conventional neurosurgery relies on neurosurgeons using medical imaging for preoperative surgical planning, such as assessing the incision location. During surgery, the operating neurosurgeon must rely on personal experience to locate the anatomical structures of the brain to gradually remove the lesion.

Different neurosurgeons having different experiences and techniques may choose different surgical paths. In fact, different surgical paths may pass through different cerebral tissues, and any surgical path passing through cerebral tissue causes some degree of brain damage, so different surgical paths naturally cause different degrees of brain damage. In other words, the success of brain surgery often depends on the experience and skill of the neurosurgeon.

According to above-mentioned contents, what is needed is to develop an improved solution to solve the conventional problem that a surgical path is determined based on neurosurgeon's subjective judgment and may be not the objectively-optimal path.

### SUMMARY

An objective of the present invention is to disclose a device of integrating an external coordinate system and a brain model to generate a biopsy path and a method thereof, to solve the conventional problem that a surgical path is determined based on neurosurgeon's subjective judgment and may be not the objectively-optimal path.

To achieve the objective, the present invention provides a device of integrating an external coordinate system and a brain model to generate a biopsy path, and the device includes an image obtaining module, an image registration module, a signal extraction module, a model construction module, a coordinate system definition module, an input module, a path calculation module, a display module, and a target marking module. The image obtaining module is configured to obtain multiple brain images of a target person, wherein the brain images include a brain structure image, a cerebral artery image, a cerebral vein image, and a brain function template. The image registration module is configured to perform registration on the cerebral artery image, the cerebral vein image and the brain function template with the brain structure image, to generate a registration integration image. The signal extraction module is configured to use tissue segmentation algorithms corresponding to different cerebral tissues to extract binary mask images of the cerebral tissues from the registration integration image, respectively, wherein the cerebral tissues comprise skin surfaces, gray matter surfaces, white matter surfaces, cerebral arteries, or cerebral veins. The model construction module is configured to reconstruct 3D models based on the binary mask images. The coordinate system definition module is configured to define a coordinate origin, coordinate quadrants, and a coordinate range based on multiple marker points on a skin surface model in the 3D models, to establish an external coordinate system. The input module is configured to select a target area in the 3D models and set a first biopsy point. The path calculation module is configured to calculate a first biopsy coordinate of the first biopsy point in the external coordinate system, calculate a first biopsy path in the 3D models based on position information of the target area in the 3D models and the first biopsy coordinate, and calculate related data of the first biopsy path, wherein the related data of the first biopsy path comprises a path distance, an area that the path passed through, a volume of gray matter that the path passed through, a volume of the white matter that the path passed through, a volume of arteries that the path passed through, or a volume of the veins that the path passed through. The display module is configured to display the 3D models and the related data of the first biopsy path. The target marking module is configured to mark the first biopsy path in the 3D models, to make the display module display the 3D models with the marked first biopsy path.

To achieve the objective, the present invention provides a method of integrating an external coordinate system and a brain model to generate a biopsy path, and the method includes steps of: obtaining multiple brain images of a target person, wherein the brain images comprise a brain structure image, a cerebral artery image, a cerebral vein image, and a brain function template; performing registration on the cerebral artery image, the cerebral vein image, and the brain function template with the brain structure image, to generate a registration integration image; using tissue segmentation algorithms corresponding to different cerebral tissues to extract binary mask images of cerebral tissue from the registration integration image, respectively, wherein the cerebral tissues comprise skin surface, gray matter surface, white matter surface, cerebral arteries, or cerebral veins; reconstructing 3D models based on the binary mask images, wherein the 3D models comprise a 3D model of the cerebral tissues; defining a coordinate origin, coordinate quadrants, and a coordinate range based on multiple marker points on a skin surface model in the 3D models, to establish an external coordinate system; selecting a target area in the 3D models, and set a first biopsy point; calculating a first biopsy coordinate of the first biopsy point in the external coordinate system, calculating a first biopsy path in the 3D models based on position information of the target area in the 3D models and the first biopsy coordinate, and calculating related data of the first biopsy path, wherein the related data of the first biopsy path comprises a path distance, an area that the path passed through, a volume of gray matter that the path passed through, a volume of the white matter that the path passed through, a volume of arteries that the path passed through, or a volume of the veins that the path passed through; displaying the 3D models and the related data of the first biopsy path, and mark the first biopsy path in the 3D models.

According to the above-mentioned device and method of the present invention, the difference between the present invention and the conventional technology is that, in the present invention, through registration of the brain images of the target person, the registration integration image is generated, the binary mask images of different cerebral tissues are extracted from the registration integration image, the 3D models is reconstructed based on the binary mask images of the cerebral tissues, the external coordinate system on the skin surface model in the 3D models is established; after the target area in the 3D models is selected and the biopsy point is set, and the biopsy path in the 3D models is calculated based on the position information of the target area in the 3D models and the coordinate of the biopsy point in the external coordinate system, and the biopsy path in the 3D models is marked. Therefore, the conventional problem can be solved, and the technical effect of objectively evaluating a lower-risk biopsy path based on the brain model of the patient can be achieved.

### BRIEF DESCRIPTION OF THE DRAWINGS

The structure, operating principle and effects of the present invention will be described in detail by way of various embodiments which are illustrated in the accompanying drawings.

FIG. 1 is a schematic view of components of a device of integrating an external coordinate system and a brain model to generate a biopsy path, according to the present invention.

FIG. 2 is a schematic view of modules of a device of integrating an external coordinate system and a brain model to generate a biopsy path, according to the present invention.

FIG. 3 is a schematic view of a brain image, according to an embodiment of the present invention.

FIG. 4 is a schematic view of an external coordinate system, according to an embodiment of the present invention.

FIG. 5A is a flowchart of a method of integrating an external coordinate system and a brain model to generate a biopsy path, according to the present invention.

FIG. 5B is a flowchart of an operation of selecting a biopsy point based on related data of a biopsy path, according to the present invention.

FIG. 6 is a schematic view of 3D models of cerebral tissues, according to an embodiment of the present invention.

FIG. 7A is a schematic view of a first biopsy path, according to an embodiment of the present invention.

FIG. 7B is a schematic view of a second biopsy path, according to an embodiment of the present invention.

### DETAILED DESCRIPTION

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions, and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first,’ ‘second,’ ‘third,’ and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include,” and variations such as “comprises,” “comprising,” “includes,” or “including,” will be acknowledged to imply the inclusion of stated elements but not the exclusion of any other elements.

In the present invention, multiple two-dimensional brain images are used to reconstruct three-dimensional (3D) models and define an external coordinate system; furthermore, the 3D models are integrated with the external coordinate system to mark a path from a biopsy point of interest to a target area in the brain model.

The brain images used in the present invention are obtained through non-invasive imaging techniques such as magnetic resonance imaging (MRI), computed tomography (CT), and angiography. The brain images can include, but is not limited to, images of brain structures cerebral tissues, or cerebrovascular images. The external coordinate system in the present invention represents a position of any point on a skin surface.

The device of the present invention can be implemented by a computing apparatus. The computing apparatus mentioned in the present invention can include, but not limited to, one or more processing module, one or more memory module, and a bus connected to different hardware components including the memory module and the processing module. Through the multiple hardware components, the computing apparatus can load and execute the operating system, so that the operating system runs on the computing apparatus and executes software or programs. In addition, the computing apparatus can include an outer shell, and the above-mentioned hardware component are disposed in the outer shell.

The bus of the mentioned computing apparatus in the present invention can include at least one type of bus, for example, the bus can include at least one of a data bus, an address bus, a control bus, an expansion bus, and a local bus. The bus of a computation device can include, but not limited to, a parallel bus such as an industry standard architecture (ISA) bus, a peripheral component interconnect (PCI) bus, a video electronics standards association (VESA) local bus, or a serial bus such as a USB, or a PCI express (PCI-E/PCIe) bus.

The processing module of the computing apparatus of the present invention is coupled with the bus. The processing module includes a register group or a register space. The register group or the register space can be completely set on the processing chip of the processing module or can be all or partially set outside the processing chip and is coupled to the processing chip through dedicated electrical connection and/or a bus. The processing module can be a central processing unit, a microprocessor, or any suitable processing component. If the computing apparatus is a multi-processor apparatus, that is, the computing apparatus includes processing modules, and the processing modules can be all the same or similar, and coupled and communicated with each other through a bus. The processing module can interpret a computer instruction or a series of multiple computer instructions to perform specific operations or operations, such as mathematical operations, logical operations, data comparison, data copy/moving, to drive other hardware component, execute the operating system, or execute various programs and/or module in the computing apparatus. The computer instructions can include assembly language instructions, instruction set architecture instructions, machine instructions, machine-related instructions, microinstructions, firmware instructions, or source code or object code written in one or more programming languages. The instructions can be executed entirely on a single computing apparatus, partially on a single computing apparatus, or partially on one computing apparatus and partially on another interconnected computing apparatus. The above-mentioned programming language can be, for example, object-oriented languages such as Common Lisp, Python, C++, Objective-C, Smalltalk, Delphi, Java, Swift, C#, Perl, Ruby, as well as procedural languages like C or similar languages.

The computing apparatus usually also includes one or more chipsets. The processing module of the computing apparatus can be coupled to the chipset, or electrically connected to the chipset through the bus. The chipset includes one or more integrated circuits (IC) including a memory controller and a peripheral input/output (I/O) controller, that is, the memory controller and the peripheral input/output controller can be implemented by one integrated circuit, or implemented by two or more integrated circuits. Chipsets usually provide I/O and memory management functions, and multiple general-purpose and/or dedicated-purpose registers, timers. The above-mentioned general-purpose and/or dedicated-purpose registers and timers can be coupled to or electrically connected to one or more processing modules to the chipset for being accessed or used. In an embodiment, the chipset can be a portion of the processing module.

The processing module of the computing apparatus can also access the data stored in the memory module and mass storage area installed on the computing apparatus through the memory controller. The above-mentioned memory modules include any type of volatile memory and/or non-volatile memory (NVRAM), such as Static Random Access Memory (SRAM), Dynamic Random Access Memory (DRAM), Read-Only Memory (ROM), or Flash memory. The above-mentioned mass storage area can include any type of storage device or storage medium, such as hard disk drives, optical discs, flash drives, memory cards, and solid state disks (SSD), or any other storage device. In other words, the memory controller can access data stored in static random access memory, dynamic random access memory, flash memory, hard disk drives, and solid state drives.

The processing module of the computing apparatus can also connect and communicate with peripheral devices and interfaces including peripheral output devices, peripheral input devices, communication interfaces, or data/signal receivers through the peripheral I/O controller and the peripheral I/O bus. The peripheral input device can be any type of input device, such as a keyboard, mouse, trackball, touchpad, or joystick. The peripheral output device can be any type of output device, such as a display, or a printer; the peripheral input device and the peripheral output device can also be the same device such as a touch screen. The communication interface can include a wireless communication interface and/or a wired communication interface. The wireless communication interface can include the interface capable of supporting wireless local area networks (such as Wi-Fi, Zigbee, etc.), Bluetooth, infrared, and near-field communication (NFC), 3G/4G/5G and other mobile communication network (cellular network) or other wireless data transmission protocol; the wired communication interface can be an Ethernet device, a DSL modem, a cable modem, an asynchronous transfer mode (ATM) devices, or optical fiber communication interfaces and/or components. The data/signal receiver can include a GPS receiver or physiological signal receiver. The physiological signals received by the physiological signal receiver include, but are not limited to, heartbeat, blood oxygen levels, and so on. The processing module can periodically poll various peripheral devices and interfaces, so that the computing apparatus can input and output data through various peripheral devices and interfaces, and communicate with another computing apparatus having the above-mentioned hardware components.

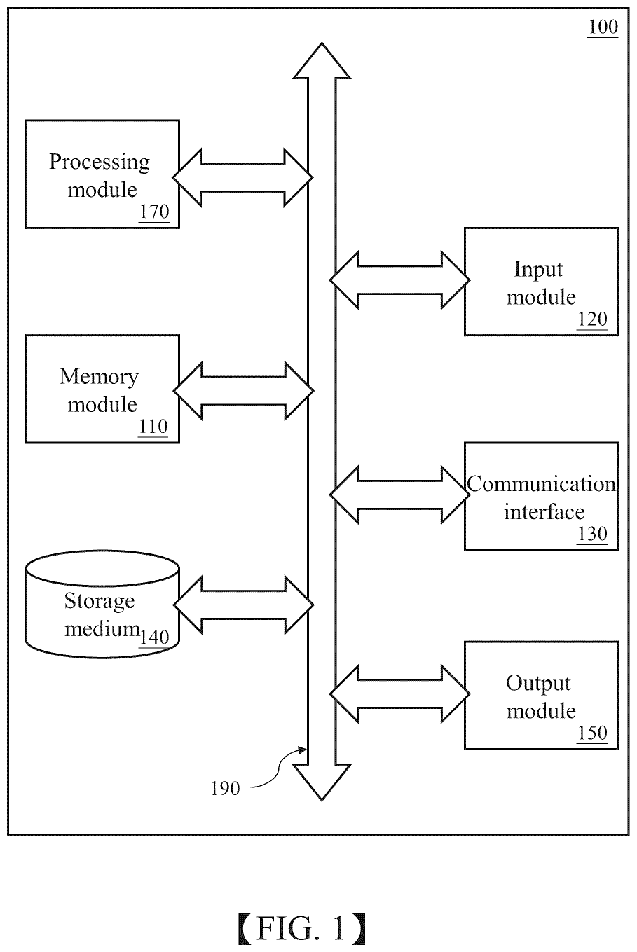

The implementation of the present invention is explained using a schematic diagram of the components of a device of integrating an external coordinate system and a brain model to generate a biopsy path with reference to FIG. 1. As shown in FIG. 1, a device 100 includes a memory module 110, an input module 120, a communication interface 130, a storage medium 140, an output module 150, a processing module 170, and a bus 190. The memory module 110, the communication interface 130, the storage medium 140, and the processing module 170 are connected via the bus 190 for mutual communication.

The memory module 110 store at least one set of computer instructions.

The input module 120 is configured to provide input data and store the input data in the storage medium 140 or directly supply the input data to the processing module 170. More specifically, the input module 120 can provide a user to select a brain area of interest (the selected brain area is also called a target area in the present invention), and to set a biopsy point on the skin surface.

The communication interface 130 is connected to a network device such as an external network storage device or a server (not shown in FIG. 1) and configured to request and download data from the connected network device. The communication interface 130 transmits the data generated by the processing module 170 to an external network storage device, a server, a display device, or a personal computer.

The storage medium 140 is configured to store data received by the communication interface 130 and also store data provided to the processing module 170, as well as the data generated by the processing module 170.

The output module 150 is configured to output the data generated by the processing module 170. More specifically, the output module 150 can include any of the aforementioned peripheral output device, thereby allowing the output of images and/or data generated by the processing module 170 through display or printing.

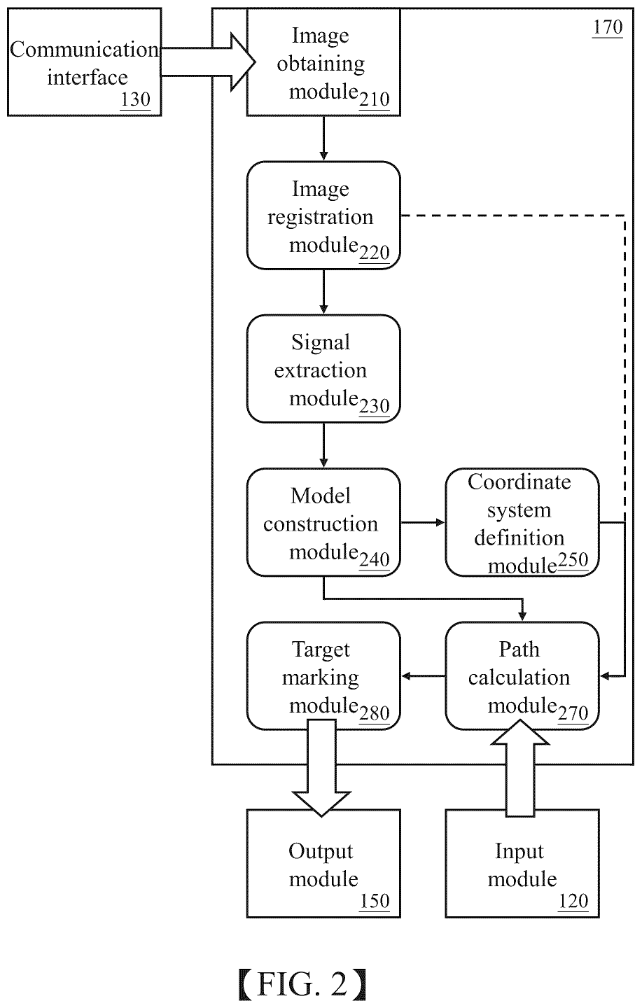

As shown in FIG. 2, which shows modules of the device of integrating the external coordinate system and the brain model to generate the biopsy path proposed in the present invention, the processing module 170 includes an image obtaining module 210, an image registration module 220, a signal extraction module 230, a model construction module 240, a coordinate system definition module 250, a path calculation module 270, and a target marking module 280. In some embodiments, the processing module 170 executes the computer instructions stored in the memory module 110 to generate the various modules shown in FIG.

2. In other embodiments, the modules in FIG. 2 may be implemented using one or more circuits and/or complete or partial hardware components such as chips. That is, the processing module 170 includes the hardware components that constitute the modules in FIG. 2, in other words, and the modules included in the processing module 170 may be software modules or hardware modules, without specific limitations in the present invention.

The image obtaining module 210 is configured to obtain the brain images of the target person. As shown in FIG. 3, the brain images obtained by the image obtaining module 210 includes a brain structure image, cerebral artery images, and cerebral vein images. The brain structure image is a T1 weighted image 320 obtained through magnetic resonance imaging (MRI), the cerebral artery images may be cerebral artery MRI images 330 obtained through magnetic resonance angiography (MRA) technology, or cerebral artery angiography images obtained by computed tomography angiography (CTA) technology or digital subtraction angiography; the cerebral vein images may be cerebral vein MRI images 340 obtained through magnetic resonance angiography technology or other cerebral vein images obtained by computed tomography angiography technology. The brain images obtained by the image obtaining module 210 includes a standard brain function template, such as MNI 152 standard brain template 310, the brain function template records position information of various brain segments.

The image obtaining module 210 directly reads the brain images from the storage medium 140, or can be connected to a storage device (not shown in FIG. 1 and FIG. 2) via the communication interface 130 to download the brain images, or can be connected to the imaging apparatus (such as MRI machines, CT scanners, digital subtraction angiography machines) based on an application programming interface (API) provided by the imaging apparatus that generates the brain images, to download the brain image generated by the imaging apparatus via the communication interface 130.

The image registration module 220 is configured to perform registration on the brain images obtained by the image obtaining module 210 to generate registration integration images after registration. More specifically, the image registration module 220 performs registration on the cerebral artery images, the cerebral vein images, the brain function template with the brain structure image to generate the registration integration images; for example, the image registration module 220 can use linear registration functions or calculation formulas to translate and/or rotate the cerebral artery image and/or the cerebral vein image, thereby generating the cerebral artery images and the cerebral vein images that can directly correspond (map) the cerebral arteries and the cerebral veins to the brain structure image, that is, the image registration module 220 generates the cerebral artery image and the cerebral vein image that can be directly superimposed with the brain structure image. For another example, the image registration module 220 can use the nonlinear registration functions or algorithms to generate a nonlinear transformation matrix for processing the brain function template by translating, rotating, scaling, and/or deforming functions. Through the generated nonlinear transformation matrix, the brain function template can be mapped to the brain structure image, so that the brain function segments (e.g., AAL3, HCPMNP, Yeo400, etc.) defined in the brain function template can be combined with the brain structure image via the nonlinear transformation matrix. The image registration module 220 stores the nonlinear transformation matrix used for the nonlinear registration in the storage medium 140, for the path calculation module 270 to use.

The signal extraction module 230 is configured to use the tissue segmentation algorithms corresponding to different cerebral tissues to extract the binary mask images of various cerebral tissues from the registration integration image generated by the image registration module 220, respectively. The cerebral tissues mentioned in the present invention can include, but not limited to, skin surface, gray matter surface, white matter surface, cerebral arteries, or cerebral veins.

In some embodiments, the signal extraction module 230 is configured to set parameters corresponding to various cerebral tissues and use specific tissue segmentation algorithm to extract the binary mask images of the cerebral tissues such as skin surface, gray matter surface, and white matter surface from the registration integration image. The signal extraction module 230 can process the registration integration image with a predetermined arterial threshold to extract the binary arterial signal image from the registration integration image, the binary arterial signal image contains multiple voxels representing vascular signals, and these voxels representing vascular signals may be connected, the signal extraction module 230 retains the group of connected voxels with the maximum voxel connection amount (this group is called in the present invention referred to as a largest connected voxel) and removes the unretained voxels (i.e., the voxels not belonging to the largest connected voxel) from the binary arterial signal image, thereby generating a binary mask image of cerebral arteries. The signal extraction module 230 can process the registration integration image with a predetermined venous threshold to extract the binary venous signal image from the registration integration image, the binary venous signal image also contains multiple voxels representing vascular signals, and these voxels representing the vascular signals may be connected. The signal extraction module 230 removes connected voxels with a voxel size less than predetermined voxel count from the binary venous signal image to generate a binary mask image of cerebral veins. The arterial threshold (the venous threshold) can be an average value of all voxel values of the cerebral artery image (the cerebral vein image) plus two times the standard deviation, but the present invention is not limited to above-mentioned examples.

The signal extraction module 230 obtains the binary image B generated after threshold segmentation, and B (i, j, k) represents a value of a voxel in the binary image B; generally, a voxel with a value of 0 means it is background, and a voxel with a value of 1 means it is a target object, such as a blood vessel. Then, the signal extraction module 230 begins searching for connected voxels in the binary image B; generally, the signal extraction module 230 starts from a voxel (i₀, j₀, k₀) in the binary image B, when a voxel (i₀, j₀, k₀) has a value of 1 and has not been searched, the signal extraction module 230 checks whether the values of voxels adjacent to voxel (i₀, j₀, k₀) is 1, and if yes, the signal extraction module 230 continues to search another voxels adjacent to the voxel (i₀, j₀, k₀) having a value of 1 until all adjacent voxels to the voxel having a value of 1 are searched. Then, the signal extraction module 230 calculates the voxel count of the connected voxels. Afterward, the signal extraction module 230 continues to search through the unsearched voxels in the binary image B until all voxels of the binary image B have been searched. When the binary image B is a binary arterial signal image, the signal extraction module 230 selects and retains the largest connected voxel in the binary image B (i.e., values of all voxels not belonged to the largest connected voxel are set to 0); when the binary image B is a binary venous signal image, the signal extraction module 230 selects and removes the connected voxel with a size less than a predetermined voxel count from the binary image B (i.e., the values of the connected voxels with a size smaller than predetermined voxel count is set to 0). For example, the predetermined voxel count can be 1000, but the present invention is not limited to above-mentioned examples.

The model construction module 240 reconstructs 3D models based on the binary mask images obtained by the signal extraction module 230, and the 3D models reconstructed by the model construction module 240 include 3D models of various cerebral tissues, such as skin surface model, gray matter model, white matter model, cerebral arteries model, and cerebral veins model.

For example, the model construction module 240 uses surface reconstruction methods to perform calculations on the binary mask images of various cerebral tissues to establish 3D models. The surface reconstruction is performed by using a triangular mesh algorithm to reconstruct the contours of the cerebral tissues defined by the binary mask images. In some embodiments, after completing surface reconstruction, the model construction module 240 performs post-processing on the generated 3D models to improve the appearance of the 3D models. This post-processing can include, but not limited to, processes of smoothing surfaces, removing unnecessary structures, and filling holes.

The coordinate system definition module 250 is configured to establish the external coordinate system based on the skin surface model in the 3D models established by the model construction module 240. More specifically, the coordinate system definition module 250 divides the target person's skull based on multiple marker points on the skin surface model, to define a coordinate origin, coordinate quadrants, and a coordinate range based on a distance between marker points, thereby setting up the external coordinate system.

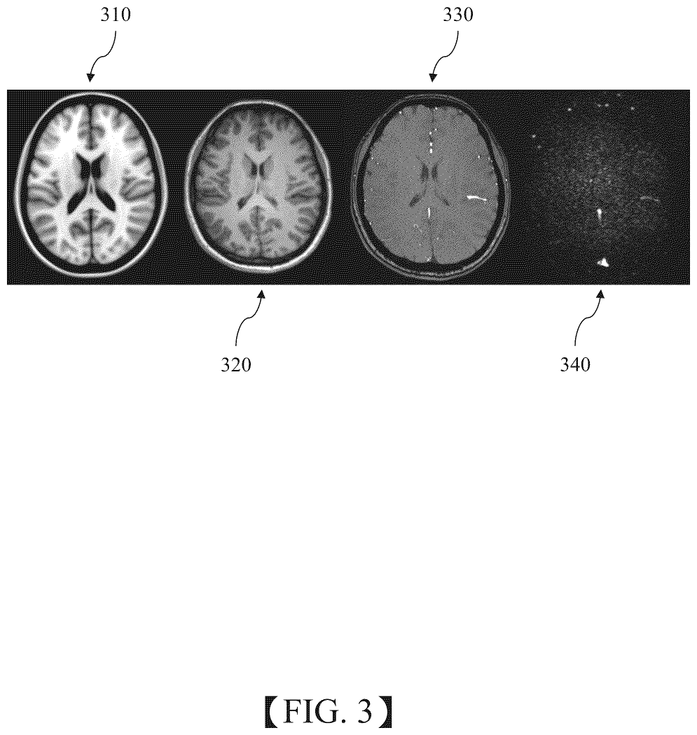

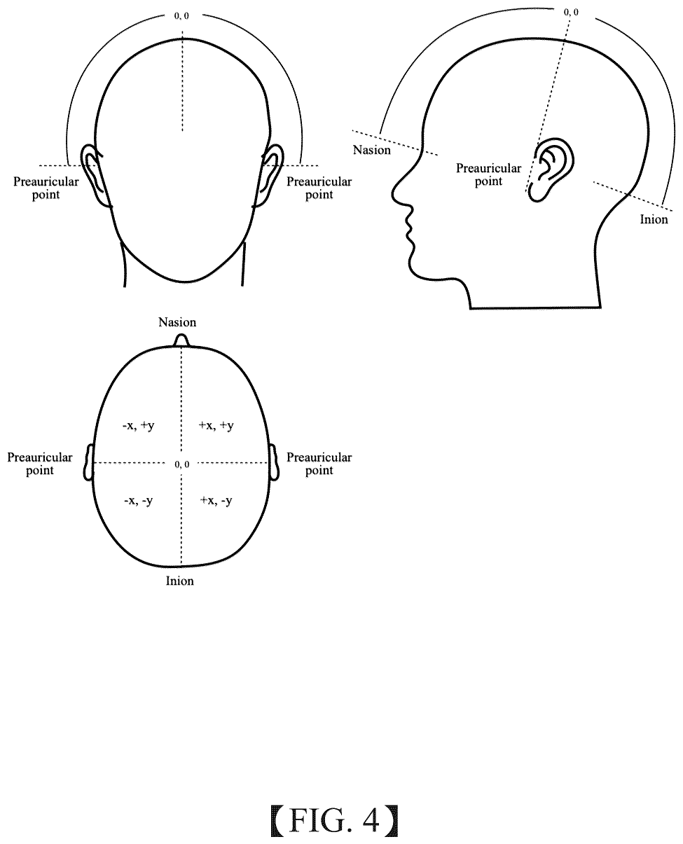

For example, as shown in FIG. 4, the coordinate system definition module 250 determines a nasion 411, an inion 412, a right preauricular side 413, and a left preauricular side 414 on the skin surface model, and uses the determined nasion 411, inion 412, right preauricular side 413, and left preauricular side 414 as the marker points. The coordinate system definition module 250 connects the nasion 411 to the inion 412, and the right preauricular side 413 to the left preauricular side 414, to define the coordinate origin 430 and the coordinate quadrants of the external coordinate system. For example, the coordinate system definition module 250 can connect the nasion 411 to the inion 412, thereby dividing the target person's skull into left and right parts; the coordinate system definition module 250 can connect the right preauricular side 413 to the left preauricular side 414, thereby dividing the target person's skull into front and back parts. The coordinate system definition module 250 defines the coordinate origin 430 as a midpoint of a line connecting the nasion 411 to the inion 412 and a midpoint of a line connecting the right preauricular side 413 to the left preauricular side 414, and defines the four quadrants of the external coordinate system based on the lines connecting the nasion 411 to the inion 412 and the right preauricular side 413 to the left preauricular side 414. For example, a coordinate of any point from the coordinate origin 430 to the right front of the target person is (+x, +y), a coordinate of any point from the coordinate origin 430 to the left front of target person is (−x, +y), a coordinate of any point from the coordinate origin 430 to the right rear of target person is (+x,−y), and a coordinate of any point from the coordinate origin 430 to the left rear of target person is (−x,−y). In some embodiments, the coordinate system definition module 250 normalizes the distances of the lines from the nasion 411 to the inion 412 and from the right preauricular side 413 to the left preauricular side 414, respectively, to define the coordinate range of the external coordinate system. For example, the coordinate range of each of X and Y axis of the external coordinate system are defined as from −1 to 1.

The path calculation module 270 calculates the coordinates of the biopsy points, including the first biopsy point and the second biopsy point, provided by the input module 120 corresponding to the external coordinate system generated by the coordinate system definition module 250, and/or coordinates of the biopsy points in the 3D brain model generated by the model construction module 240. The coordinates of the biopsy points in the 3D models can be represented as a first biopsy coordinate and a second biopsy coordinate in the present invention. For example, when the input module 120 is used to set the biopsy point in the external coordinate system, the path calculation module 270 can calculate the coordinates of the biopsy point on the external coordinate system based on the distance from the set biopsy point to the X and Y axes of the external coordinate system, and map the biopsy point to the skin surface model based on the transformation matrix or transformation formula used to establish the skin surface model of the 3D models by the model construction module 240; based on coordinates of the mapped location of the biopsy point on the skin surface model that corresponds to the on the X, Y, and Z axes of the 3D models, the path calculation module 270 can obtain the coordinates of the biopsy point in the 3D models. When the input module 120 provides a user to set a biopsy point on the skin surface model in the 3D models, the path calculation module 270 obtains the coordinates of the biopsy point in the external coordinate system based on the respective distances from the biopsy point to the X, Y, and Z axes of the 3D models, and maps the biopsy point to the external coordinate system based on the transformation matrix or transformation formula used to establish the skin surface model of the 3D models by the biopsy point model construction module 240, and calculates coordinates of the mapped point (that is, the biopsy point) corresponding to the X Y axis of the external coordinate system based on the mapped position of the biopsy point on the skin surface model. For example, in a condition that that the coordinates of the biopsy point in the 3D models is (104, 218, 170), when the biopsy point is mapped to the normalized external coordinate system where the coordinates of any point from the coordinate origin 430 to the nasion 411 is in the range of 0 to 1, and when the distance between the coordinate origin 430 and the nasion 411 is 10 cm, and the mapped point is on the X-axis and the distance from the mapped point to the Y-axis is 7 cm, the coordinate of the mapped point in the external coordinate system would be (0, 0.7).

The path calculation module 270 also obtains the position information of the target area, which is selected through the input module 120, in the 3D models created by the model construction module 240. The position information obtained by the path calculation module 270 is typically the coordinate in the 3D models, but the present invention is not limited to above-mentioned examples. In an embodiment, the path calculation module 270 obtains the nonlinear transformation matrix generated by the image registration module 220, and uses the obtained nonlinear transformation matrix to compute and generate the position information of the target area mapped to the 3D models based on the location data of the target area in the brain function template.

Based on the position information of the selected target area provided by the input module 120 in the 3D models created by the model construction module 240 and the biopsy coordinate of the biopsy point input by the input module 120 in the 3D models, the path calculation module 270 calculates the biopsy paths in the 3D models that can include a first biopsy path and a second biopsy path. More specifically, the path calculation module 270 calculates the biopsy path based on a connection line between the position information of the target area and the biopsy coordinate, as well as a probe radius input by the input module 120. For example, in a condition that the position information of the target area is a coordinate as P₁=(x₁, y₁, z₁), and the biopsy coordinate is P₂=(x₂, y₂, z₂), the path calculation module 270 connects the coordinates of the position information and the biopsy coordinate as an axis, defines a cylinder with the starting and ending points being the coordinates of the position information and the biopsy coordinate and the probe radius (r). The defined cylinder is the biopsy path.

The axis equation of the cylinder defined by the path calculation module 270 is:

$$\{ \begin{matrix}x ( t ) = {x}_{1} + ( {x}_{2} - {x}_{1} ) \cdot t \\ y ( t ) = {y}_{1} + ( {y}_{2} - {y}_{1} ) \cdot t \\ z ( t ) = {z}_{1} + ( {z}_{2} - {z}_{1} ) \cdot t\end{matrix}$$

where t is a parameter ranging from 0 to 1; when t=0, the coordinate of the target area on the axis is P₁; when t=1, the biopsy coordinate on the axis is P₂.

The path calculation module 270 also calculates the related data of the biopsy path. The related data of the biopsy path in the present invention can include, but not limited to, a path distance, an area that the path passed through, a volume of gray matter that the path passed through, a volume of the white matter that the path passed through, a volume of arteries that the path passed through, and a volume of the veins that the path passed through.

For example, points (x, y, z) on the surface of the cylinder satisfy the following cylinder equation condition: (x−x(t))²+(y−y(t))²+(z−z(t))² =r²

where (x(t), y(t), z(t)) is the point on the cylinder's axis and r is the radius of the cylinder.

The path calculation module 270 obtains the coordinates of each voxel in the 3D models, substitutes the coordinates of voxels into the cylinder equation. In a condition that the coordinates of a certain voxel is (i, j, k), when the coordinate of the voxel satisfy the cylinder equation (i−x(t))²+(j−y(t))²+(k-−z(t))²≤r², the voxel is inside the cylinder; when the coordinates of the voxel do not satisfy the cylinder equation, the voxel is outside the cylinder. In this way, the path calculation module 270 performs calculations using the coordinates of voxels in the 3D models to determine which voxels in the 3D models are within the range of the cylinder based on the cylinder equation, to generate a binary image of the voxels within the range of the cylinder. In the present invention, the generated binary image is referred to as the binary path image.

The path calculation module 270 superimposes the binary path image with the binary mask images of cerebral tissues to calculate the related data of the biopsy path, respectively. For example, the path distance is calculated by d=\sqrt{(x₂−x₁)²+(y₂−y₁)²+(z₂−z₁)²} ; the area that the path passed through is calculated by multiplying the binary path image with the brain segment templates to obtain products, correspond the product to the different brain regions based on a data table in the brain region template, wherein the corresponding brain region is the area that the path passed through; the volumes of gray matter, white matter, arterial and venous that the path passed through are calculating by multiply the binary path image with the binary mask images of the gray matter, white matter, arteries, and veins obtained after tissue segmentation, and counting the remaining voxels and multiplied the counted result with unit sizes of their voxel sizes, wherein the products are the volumes of gray matter, white matter, arterial and venous that the path passed through.

The path calculation module 270 can generate a suggestion message for selecting the first biopsy point or the second biopsy point based on the related data of the first biopsy path and the second biopsy path. Generally, the path calculation module 270 prioritizes the volume of arteries and veins passed through by the biopsy path as the basis for selecting the biopsy path. For example, the path calculation module 270 first selects the biopsy path with the least artery volume; when the volumes of arteries passed through by the first biopsy path and the second biopsy path are the same, the path calculation module 270 further selects the path with the least vein volume. When the volumes of both arteries and veins passed through by the first biopsy path and the second biopsy path are the same are the same, the path calculation module 270 selects the biopsy path with the shorter path distance or the path with the least volume of gray matter or white matter. However, the manner of selecting one of the first biopsy point and the second biopsy point by the path calculation module 270 based on the related data of the first biopsy path and the second biopsy path is not limited to the above examples.

The target marking module 280 is configured to mark the biopsy path calculated by the path calculation module 270 in the 3D models established by the model construction module 240, so that the output module 150 can display the 3D models with the marked biopsy path. It is to be noted that, when the path calculation module 270 calculates multiple biopsy paths, the target marking module 280 can display all of at least one of the biopsy path based on a predefined or selected option.

The operation system and method of the present invention will be explained with reference to an embodiment. Please refer to FIG. 5A. FIG. 5A is a flowchart of a method of integrating an external coordinate system and a brain model to generate a biopsy path according to the present invention. In this embodiment, the device 100 applying the present invention is a computer, but the present invention is not limited thereto.

When a neurosurgeon uses the device of the present invention, the image obtaining module 210 of the device 100 obtains the brain images of the target person (step 510). In this embodiment, the image obtaining module 210 is connected to an external storage device through the communication interface 130 of the device 100 to read the brain images based on identification data (such as the ID number or medical record number) of the target person; the brain images obtained by the image obtaining module 210 can include, but not limited to, a brain structure image, a cerebral artery image, a cerebral vein image, and a brain function template.

When the image obtaining module 210 of the device 100 obtains the brain images in the step 510, the image registration module 220 of the device 100 performs registration on the brain image to generate the registration integration image (step 520). In this embodiment, the image registration module 220 can use a registration function: antsRegistrationSyNQuick () of the open-source software ANTs (http://stnava.github.io/ANTs/) to translate and/or rotate the cerebral artery image and the cerebral vein image in the brain images to register with the brain structure image by linear registration, and use the registration function: antsRegistrationSyNQuick()of ANTs software and the nonlinear transformation matrix function: antsApplyTransforms() to perform nonlinear registration on the brain function template of the brain images with the brain structure image by translation, rotation, scaling, and deformation.

After the image registration module 220 of the device 100 completes the registration of the brain images and generates the registration integration image, the signal extraction module 230 of the device 100 extracts different binary mask images of different cerebral tissues from the registration integration image by using different tissue segmentation algorithm (step 530). In this embodiment, the signal extraction module 230 extracts different binary mask images of cerebral tissues (such as the skin surface, gray matter, and white matter) from the registration integration image by using the FAST() function in the FSL software, and the signal extraction module 230 extracts the binary arterial signal image and the binary venous signal image from the registration integration image by using the predetermined arterial threshold and venous threshold, respectively, and removes non-largest connected voxels from the binary arterial signal image to generate the binary cerebral artery mask image, and remove connected voxels having sizes smaller than predetermined voxel count (e.g., 1000) from the binary venous signal image to generate the binary cerebral vein mask image.

After the signal extraction module 230 of the device 100 obtains the binary mask images of the cerebral tissues, the model construction module 240 of the device 100 reconstructs 3D models of the cerebral tissues (the 3D models including a skin surface model, a gray matter model, a white matter model, a cerebral arteries model, and a cerebral veins model) based on the binary mask images of the cerebral tissues (step 540). In this embodiment, the model construction module 240 reconstructs the 3D models of the cerebral tissues through surface reconstruction based on the binary mask images of the cerebral tissues; after reconstructing the 3D models of the cerebral tissues, the model construction module 240 performs post-processing (such as smoothing surfaces, removing unnecessary structures, and filling holes) on the 3D models and then combines the reconstructed 3D models of the cerebral tissues into the 3D models. Please refer to FIG. 6, which illustrates a 3D skin surface model 610, a 3D gray matter model 630, a 3D artery model 650, a 3D vein model 660, and 3D models 600 including gray matter surface, arteries, and veins.

After the model construction module 240 of the device 100 establishes the 3D models of the patient, the coordinate system definition module 250 of the device 100 establishes the external coordinate system based on the skin surface model in the 3D models, (step 550). In this embodiment, the coordinate system definition module 250 determines the nasion 411, the inion 412, the right preauricular side 413, and the left preauricular side 414 on the skin surface model 400, uses the determined nasion 411, inion 412, right preauricular side 413, and left preauricular side 414 as the marker points, and connects the nasion 411 to the inion 412 and the right preauricular side 413 to the left preauricular side 414, so that the midpoint of the connection line between the nasion 411 and the inion 412 intersects with the midpoint of the connection line between the right preauricular side 413 and the left preauricular side 414, thereby defining the coordinate origin 430 of the external coordinate system at the intersection point and coordinate quadrants. The coordinate system definition module 250 also normalizes the distance between the nasion 411 and the inion 412 and the distance between the right preauricular side 413 and the left preauricular side 414, respectively, thereby defining the coordinate ranges of the X-axis and Y-axis of the external coordinate system as from −1 to 1.

When the coordinate system definition module 250 of the device 100 establishes the external coordinate system, the input module 120 of the device 100 allows a user (neurosurgeon) to select the target area in the 3D models and enables the user to set the first biopsy point on the skin surface model in the 3D models created in the model construction module 240 of the device 100 (step 560). In this embodiment, the target area can be the right hippocampus.

After the input module 120 of the device 100 provides the user to select the target area for setting the first biopsy point (step 560), the path calculation module 270 of the device 100 calculates the first biopsy coordinate, such as (0.92,-0.09), of the first biopsy point set by the input module 120 of the device 100 on the external coordinate system defined by the coordinate system definition module 250 of the device 100. Based on the calculated first biopsy coordinate, and the target area selected by the input module 120 of the device 100 in the 3D models created by the model construction module 240 of the device 100, the path calculation module 270 determines the first biopsy path in the 3D models and calculates the related data of the first biopsy path (step 570). In this embodiment, the path calculation module 270 calculates the position information of the target area in the 3D models based on the nonlinear transformation matrix generated by the image registration module 220 of the device 100 during registration of the brain function template. The coordinates of the hippocampus in the 3D models can be (21, 1, 19), and the path calculation module 270 can calculate a cylindrical first biopsy path based on the connection line between the position information and the first biopsy coordinate and the input probe radius provided by the input module 120, generate the binary path image of voxels within the range of the first biopsy path, and superimpose the binary path image with the binary mask images of cerebral tissues such as gray matter, white matter, arteries, and veins, thereby calculating the path distance, the area that the path passed through, and the related data of gray matter, white matter, arteries, and veins along the first biopsy path, respectively.

After the path calculation module 270 of the device 100 calculates the first biopsy path and the related data of the first biopsy path, the target marking module 280 of the device 100 displays the 3D models created by the model construction module 240 of the device 100 and the first biopsy path and the related data of the first biopsy path calculated by the path calculation module (step 579). In this embodiment, the target marking module 280 can display a screen as shown in FIG. 7A through the output module 150 of the device 100. FIG. 7A shows a 3D skin surface model 710, a first biopsy point 715, and a coordinate (0.92,-0.09) of the first biopsy point 715 in the external coordinate system, 3D models 720, a target area 731, and a coordinate (21, 1, 19) of the target area 731 in the 3D models 720, a first biopsy path 735, and related data 741 of the first biopsy path 735.

In the above embodiment, as shown in the flow of FIG. 5B, after the path calculation module 270 of the device 100 calculates the first biopsy path and the related data of the first biopsy path, the input module 120 of the device 100 allows the user (neurosurgeon) to set the second biopsy point again on the 3D models created by the model construction module 240 of the device 100 (step 565), and the path calculation module 270 similarly calculates a second biopsy coordinate, such as (0.87, 0.08), of the second biopsy point in the external coordinate system, determines the second biopsy path in the 3D models based on the calculated second biopsy coordinate and the position information of the target area in the 3D models, and calculates the related data of the second biopsy path (step 580). In this embodiment, the target marking module 280 of the device 100 can also display a screen as shown in FIG. 7B through the output module 150 of the device 100. FIG. 7B shows the 3D skin surface model 710, a second biopsy point 716, and a coordinate (0.87, 0.08) of the second biopsy point 716 in the external coordinate system, the 3D models 720, the target area 731, and the coordinates (21, 1, 19) of the target area 731 in the 3D models 720, a second biopsy path 736, and related data 742 of the second biopsy path 736.

After the path calculation module 270 of the device 100 determines the related data of the first biopsy path and the second biopsy path (steps 570 and 580), the path calculation module 270 generates the suggestion message of selecting the first biopsy point or the second biopsy point based on the related data of the first biopsy path and the second biopsy path (step 590). In this embodiment, the path calculation module 270 selects the first biopsy point 715 or the second biopsy point 716 based on whether the first biopsy path/the second biopsy path and cerebrovascular (arteries and/or veins) are crossed. When the second biopsy path 736 does not cross arteries and veins but the first biopsy path 735 does, it indicates that the surgical risk of the second biopsy path 736 is lower than that of the first biopsy path 735. Therefore, the path calculation module 270 generates the suggestion message of selecting the second biopsy point 716 for surgery.

With the solution of the present invention, neurosurgeons can plan and assess the risks of a biopsy path for surgery, thereby selecting an appropriate biopsy path. Furthermore, with the external coordinate system, neurosurgeons can even use the biopsy point from surgical planning to compute and plan the coordinate of the biopsy point in the patient's actual anatomical space, and use it during treatment.

According to above-mentioned contents, the difference between the present invention and the conventional technology is that, in the present invention, through registration of the brain images of the target person, the registration integration image is generated, the binary mask images of different cerebral tissues are extracted from the registration integration image, the 3D models are reconstructed based on the binary mask images of the cerebral tissues, the external coordinate system on the skin surface model in the 3D models is established; after the target area in the 3D models is selected and the biopsy point is set, and the biopsy path in the 3D models is calculated based on the position information of the target area in the 3D models and the coordinate of the biopsy point in the external coordinate system, and the biopsy path in the 3D models is marked.

With the above-mentioned solution of the present invention, the conventional problem that a surgical path is determined based on neurosurgeon's subjective judgment can be solved, and the technical effect of objectively evaluating a lower-risk biopsy path based on the brain model of the patient can be achieved.

Furthermore, the method of integrating external coordinate system and brain model to generate biopsy path of the present invention can be implemented by hardware, software or a combination thereof, and can be implemented in a computer system by a centralization manner, or by a distribution manner of different components distributed in several interconnected computer systems.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A method of integrating external coordinate system and brain model to generate biopsy path, applied to a device, comprising:
obtaining multiple brain images of a target person, wherein the brain images comprise a brain structure image, a cerebral artery image, a cerebral vein image, and a brain function template;
performing registration on the cerebral artery image, the cerebral vein image, and the brain function template with the brain structure image, to generate a registration integration image;
using tissue segmentation algorithms corresponding to different cerebral tissues to extract binary mask images of cerebral tissue from the registration integration image, respectively, wherein the cerebral tissues comprise skin surface, gray matter surface, white matter surface, cerebral arteries, or cerebral veins;
reconstructing 3D models based on the binary mask images, wherein the 3D models comprise a 3D model of the cerebral tissues;
defining a coordinate origin, coordinate quadrants, and a coordinate range based on multiple marker points on a skin surface model in the 3D models, to establish an external coordinate system;
selecting a target area in the 3D models, and set a first biopsy point;
calculating a first biopsy coordinate of the first biopsy point in the external coordinate system, calculating a first biopsy path in the 3D models based on position information of the target area in the 3D models and the first biopsy coordinate, and calculating related data of the first biopsy path, wherein the related data of the first biopsy path comprises a path distance, an area that the first biopsy path passed through, a volume of gray matter that the first biopsy path passed through, a volume of the white matter that the first biopsy path passed through, a volume of arteries that the first biopsy path passed through, or a volume of the veins that the first biopsy path passed through; and
displaying the 3D models and the related data of the first biopsy path, and mark the first biopsy path in the 3D models.

2. The method of integrating external coordinate system and brain model to generate biopsy path according to claim 1, after the step of calculating the related data of the first biopsy path, further comprising:
setting a second biopsy point, calculating a second biopsy coordinate of the second biopsy point in the external coordinate system, calculating a second biopsy path based on the second biopsy coordinate and the position information, calculating the related data of the second biopsy path, and generating and displaying a suggestion message for selecting the first biopsy point or the second biopsy point based on the related data of the first biopsy path and the second biopsy path.

3. The method of integrating external coordinate system and brain model to generate biopsy path according to claim 1, wherein the step of defining the coordinate origin, the coordinate quadrants, and the coordinate range based on the multiple marker points on the skin surface model in the 3D models to establish the external coordinate system, comprises:
determining a nasion, an inion, a right preauricular side, and a left preauricular side on the skin surface model as the marker points, connecting the nasion to the inion, and the right preauricular side to the left preauricular side to define the coordinate origin and the coordinate quadrants of the external coordinate system, and normalizing connection distances between the nasion and the inion and between the right preauricular side and the left preauricular side to define the coordinate range of the external coordinate system.

4. The method of integrating external coordinate system and brain model to generate biopsy path according to claim 1, wherein the step of using the tissue segmentation algorithm corresponding to the different cerebral tissues to extract the binary mask images of the cerebral tissues from the registration integration image, comprises:
performing an operation on the registration integration image based on an arterial threshold to generate a binary arterial signal image;
retaining a largest connected voxel in the binary arterial signal image, to generate binary cerebral artery mask images of the binary mask images;
performing an operation on the registration integration image based on a venous threshold, to generate a binary vein signal image; and
removing all connected voxels of the binary venous signal images that have a size lower than a predetermined voxel count, to generate binary cerebral vein mask images of the binary mask images.

5. The method of integrating external coordinate system and brain model to generate biopsy path according to claim 1, wherein the step of calculating the first biopsy path in the 3D models based on the position information of the target area in the 3D models and the first biopsy point, and calculating the related data of the first biopsy path, comprises:
obtaining a probe radius, calculating the first biopsy path based on the probe radius and a connection line between the position information and the first biopsy point, generating a binary path image based on voxels of the first biopsy path, and superimposing the binary path image with the binary mask images of the cerebral tissues to calculate the related data of the first biopsy path, respectively.

6. A device of integrating an external coordinate system and a brain model to generate a biopsy path, wherein the device comprises:
an image obtaining module, configured to obtain multiple brain images of a target person, wherein the brain images comprise a brain structure image, a cerebral artery image, a cerebral vein image, and a brain function template;
an image registration module, configured to perform registration on the cerebral artery image, the cerebral vein image and the brain function template with the brain structure image, to generate a registration integration image;
a signal extraction module, configured to use tissue segmentation algorithms corresponding to different cerebral tissues to extract binary mask images of the cerebral tissues from the registration integration image, respectively, wherein the cerebral tissues comprise skin surfaces, gray matter surfaces, white matter surfaces, cerebral arteries, or cerebral veins;
a model construction module, configured to reconstruct 3D models based on the binary mask images;
a coordinate system definition module, configured to define a coordinate origin, coordinate quadrants, and a coordinate range based on multiple marker points on a skin surface model in the 3D models, to establish an external coordinate system;
an input module, configured to select a target area in the 3D models and set a first biopsy point;
a path calculation module, configured to calculate a first biopsy coordinate of the first biopsy point in the external coordinate system, calculate a first biopsy path in the 3D models based on position information of the target area in the 3D models and the first biopsy coordinate, and calculate related data of the first biopsy path, wherein the related data of the first biopsy path comprises a path distance, an area that the first biopsy path passed through, a volume of gray matter that the first biopsy path passed through, a volume of the white matter that the first biopsy path passed through, a volume of arteries that the first biopsy path passed through, or a volume of the veins that the first biopsy path passed through;
a display module, configured to display the 3D models and the related data of the first biopsy path; and
a target marking module, configured to mark the first biopsy path in the 3D models, to make the display module display the 3D models with the marked first biopsy path.

7. The device of integrating external coordinate system and brain model to generate biopsy path according to claim 6, wherein the input module configured to set a second biopsy point, calculate a second biopsy coordinate of the second biopsy point in the external coordinate system, calculate a second biopsy path based on the second biopsy coordinate and the position information, calculating the related data of the second biopsy path, and generate and display a suggestion message for selecting the first biopsy point or the second biopsy point based on the related data of the first biopsy path and the second biopsy path.

8. The device of integrating external coordinate system and brain model to generate biopsy path according to claim 6, wherein the coordinate system definition module determines a nasion, an inion, a right preauricular side, and a left preauricular side on the skin surface model as the marker points, connects the nasion to the inion, and the right preauricular side to the left preauricular side to define the coordinate origin and the coordinate quadrants of the external coordinate system, and normalizes connection distances between the nasion and the inion and between the right preauricular side and the left preauricular side to define the coordinate range of the external coordinate system.

9. The device of integrating external coordinate system and brain model to generate biopsy path according to claim 6, wherein the signal extraction module performs an operation on the registration integration image based on an arterial threshold to generate a binary arterial signal image, retains a largest connected voxel in the binary arterial signal image, to generate binary cerebral artery mask images of the binary mask images, performs an operation on the registration integration image based on a venous threshold, to generate a binary vein signal image, and removes all connected voxels of the binary venous signal images that have a size lower than a predetermined voxel count, to generate the binary cerebral vein mask images of the binary mask images.

10. The device of integrating external coordinate system and brain model to generate biopsy path according to claim 6, wherein the input module is configured to input a probe radius, the path calculation module calculates the first biopsy path based on the probe radius and a connection line between the position information and the first biopsy point, generates a binary path image based on voxels of the first biopsy path, and superimposes the binary path image with the binary mask images of the cerebral tissues to calculate the related data of the first biopsy path, respectively.
